(19) United States
(12) Patent Application Publication
(10) Pub. No.: US 2025/0040867 A1
(43) Pub. Date: Feb. 6, 2025

(54) WEARABLE DEVICE FOR MEASUREMENT OF BRAIN ACTIVITY

(71) Applicants: Truman Pierson, New Hope, MN (US); Ali Hussain, Boynton Beach, FL (US); Christopher Fitz, Edgewood, KY (US)

(72) Inventors: Truman Pierson, New Hope, MN (US); Ali Hussain, Boynton Beach, FL (US); Christopher Fitz, Edgewood, KY (US)

(21) Appl. No.: 18/229,340

(22) Filed: Aug. 2, 2023

(51) Int. Cl. A61B 5/00; A61B 5/259

(52) U.S. Cl. CPC A61B 5/4094; A61B 5/259; A61B 5/6803

(57) ABSTRACT

A device for measurement of neurological and/or cognitive function of a user's brain to is disclosed, as well as a system for analyzing the measured activity to notify a user of such neurological and/or cognitive function for measuring brain capacity, output, tracking learning methods, seizure activity, sleep, and/or other related events to provide better understanding, efficiency, and predictability of a user's brain function.

### FIELD OF THE DISCLOSURE

The present disclosure relates to wearable devices for the measurement of cognitive function. More particularly, the present disclosure relates to wearable devices and associated applications for the measurement and analysis of cognitive function.

### BACKGROUND OF THE DISCLOSURE

An electroencephalogram (“EEG”) is used to measure real-time spontaneous electrical activity of the brain, typically via electrodes applied directly to the scalp. The electrical activity measured by an EEG can give insight on a user's cognitive function and may also be used to aid in diagnoses of psychiatric disorders, neuropsychiatric disorders, sleep disorders, epilepsy, and brain tumors. Generally, EEGs are performed by medical personnel as a diagnostic tool. However, attention to health and performance continues to increase as people turn to and rely on wearable devices to collect data for a variety of physical functions, including heart rate, physical activity, estimated caloric output, and glucose levels. Individual access to this data allows the user to make informed decisions to reach their individual goals and may also provide medical professionals with real-time patient data to facilitate diagnosis and treatment of an individual.

### SUMMARY

A device for measurement of neurological and/or cognitive function of a user's brain to is disclosed, as well as a system for analyzing the measured activity to notify a user of such neurological and/or cognitive function for measuring brain capacity, output, tracking learning methods, seizure activity, sleep, and/or other related events to provide better understanding, efficiency, and predictability of a user's brain function.

In a first aspect of the disclosure, a wearable device for measuring neurological function is disclosed, the device including a housing; at least one electrode coupled to the body; an electronics module coupled to the body and in electric communication with the electrode; and an adhesive configured to couple the wearable device to a user. The electronics module includes a microcontroller.

In another aspect of the disclosure, a system for measuring neurological function is disclosed, the system comprising: a wearable device including an electronics module having a microcontroller and user interface. The microcontroller is configured to receive data corresponding to neurological or cognitive activity of a user, compare the received data with previously saved data to determine a status of the user, and transmit the received data to the user interface. The data is collected by at least one electrode.

In yet another aspect of the disclosure, a method for identifying a seizure occurrence is disclosed, the method comprising: collecting neurological data of a user with an electrode of a wearable device; extracting a neurological feature from the data with a microcontroller; comparing the extracted feature to previously identified features with at least one of the microcontroller or a user interface; and determining, with the at least one of the microcontroller or the user interface, if the extracted feature corresponds with a seizure occurrence.

In various aspects of the disclosure, the wearable device may define a partial C-shape.

In various aspects of the disclosure, the housing may be comprised of at least three circular sections. Each one of the at least three circular sections may house an electrode.

In various aspects of the disclosure, the device may further comprise a plurality of prongs distributed along a bottom surface of the wearable device. At least one of the plurality of prongs may define a chamber containing a plunger and a quantity of adhesive. The plunger may be configured to extrude the quantity of adhesive through an opening defined by the prong to facilitate attachment of the wearable device to the user.

In various aspects of the disclosure, the wearable device may include at least three electrodes.

In various aspects of the disclosure, the housing may define an outer shell layer. The adhesive may define an adhesive layer comprised of a gel or adhesive tape. The electronics module may define an electronics module layer sandwiched between the adhesive layer and the outer shell layer.

In various aspects of the disclosure, the adhesive may be configured to maintain contact between the wearable device and the user for at least one week.

### BRIEF DESCRIPTION OF THE DRAWINGS

The above-mentioned and other features and advantages of this disclosure, and the manner of attaining them, will become more apparent and will be better understood by reference to the following description of embodiments of the invention taken in conjunction with the accompanying drawings, wherein:

FIG. 1 illustrates placement of a first embodiment of a wearable device for monitoring neurological and/or cognitive function of a user;

FIG. 2 is a perspective view of the wearable device of FIG. 1;

FIG. 3 is a bottom perspective view of the wearable device of FIG. 1;

FIG. 4 is a partially transparent view of the interior of a prong of the wearable device of FIG. 1;

FIG. 5A is a first side perspective view of a second embodiment of a wearable device for monitoring neurological and/or cognitive function of a user;

FIG. 5B is a second side perspective view of the wearable device of FIG. 5A;

FIG. 6 illustrates a placement of the wearable device of FIG. 5A for monitoring neurological and/or cognitive function of a user;

FIG. 7 is a schematic view of an electronics module and associated system including either of the wearable device of FIG. 1 and/or the wearable device of FIG. 5A;

FIG. 8 is an exemplary illustration of a user interface of the system of FIG. 7 displaying a user's cognitive capacity in relation to the user's cognitive output;

FIG. 9 is an exemplary illustration of the user interface of FIG. 8 displaying a user's cognitive capacity in relation to the user's cognitive output at the start of the user's day;

FIG. 10 is an exemplary illustration of the user interface of FIG. 8 displaying a user's cognitive capacity in relation to the user's cognitive output as the user goes about his or her day;

FIG. 11 is an exemplary illustration of the user interface of FIG. 8 displaying an exemplary user input form for tracking user activity;

FIG. 12 is an exemplary illustration of the user interface of FIG. 8 displaying a user's cognitive capacity in relation to the user's cognitive output when the user's cognitive output is reaching the user's cognitive capacity;

FIG. 13 is an exemplary illustration of the user interface of FIG. 8 displaying a timeline of the user's cognitive output;

FIG. 14 is an exemplary illustration of the user interface of FIG. 8 displaying trends of a user's cognitive efficiency; and

FIG. 15 is a flowchart of logic for determining the occurrence of a seizure event in a user.

Corresponding reference characters indicate corresponding parts throughout the several views. The exemplifications set out herein illustrate exemplary embodiments of the invention and such exemplifications are not to be construed as limiting the scope of the invention in any manner.

### DETAILED DESCRIPTION

For the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. One embodiment of the invention is shown in great detail, although it will be apparent to those skilled in the relevant art that some features that are not relevant to the present invention may not be shown for the sake of clarity.

### Wearable Devices and Related Systems

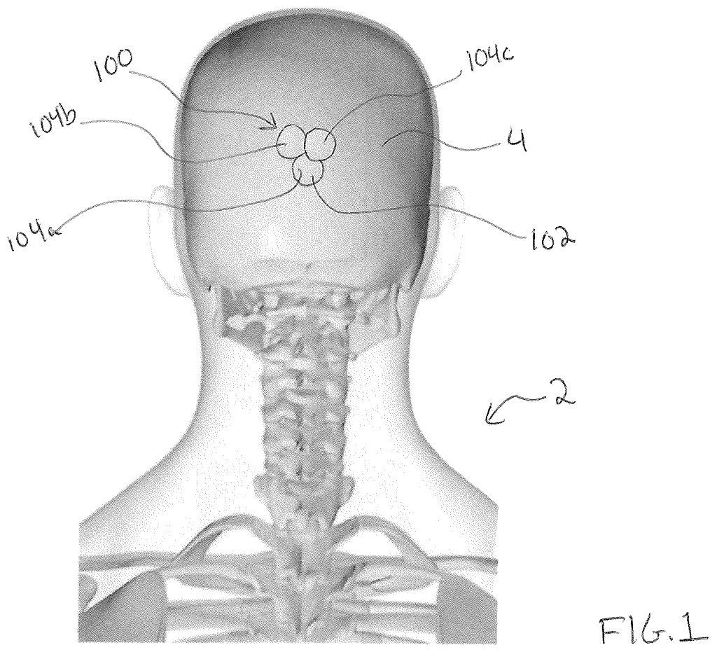

FIG. 1 illustrates a wearable scalp device 100 for measuring cognitive function. The wearable scalp device 100 includes a housing 102 having three sections 104a, 104b, 104c. Each section 104 may house an electrode 334 as discussed further herein. Each section 104 of housing 102 is illustratively circular, which may facilitate comfortable long-term wear. In other embodiments, the sections may be formed in other shapes, including edged and non-edged shapes. While sections 104 are illustratively positioned in a triangular arrangement, sections 104 may be positioned in other arrangements and with a greater or fewer number of sections. For example, in other embodiments, wearable scalp device 100 may have one, two, four, five, or more sections, which may be arranged in a linear, circular, or square shape. Other embodiments may have sections arranged in other shapes to facilitate operation of the wearable device as discussed further herein. Housing 102 further houses an electronics module 346 for operation of wearable scalp device 100 and to facilitate communication between wearable scalp device 100 and a user interface as described further herein.

Wearable scalp device 100 is preferably small in size and weight to avoid or otherwise mitigate interference with the user's daily life while in use. Wearable scalp device 100 may also be designed in a camouflaged manner for aesthetic purposes and/or to avoid detectability by an onlooker. For example, housing 102 and associated protrusions as discussed further herein may be colored to generally blend in with a user's hair or skin.

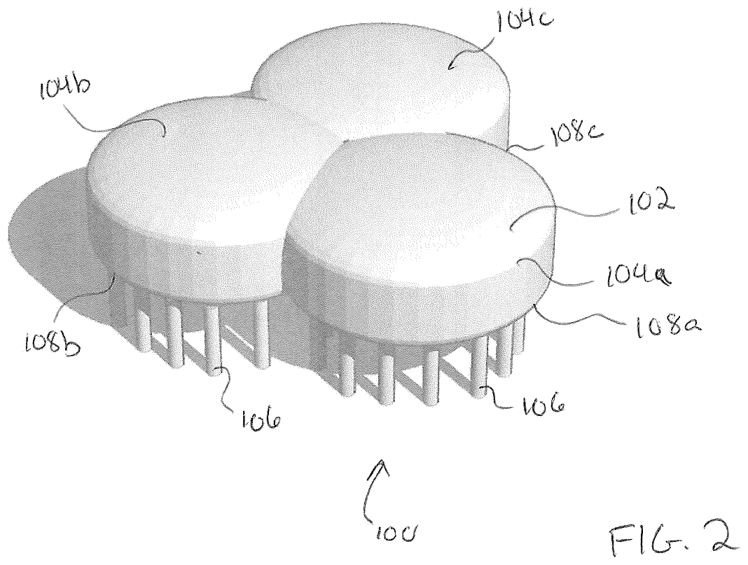

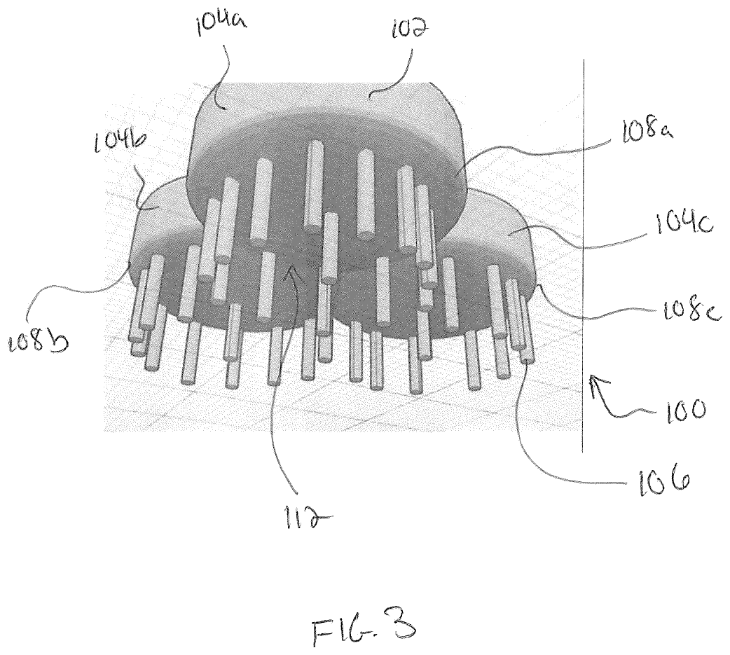

Referring additionally to FIGS. 2-3, wearable scalp device 100 includes a plurality of protrusions 106 extending from each of the sections 104. Protrusions 106 facilitate tactile connection between wearable scalp device 100 and a scalp 4 of a user 2 (FIG. 1), and can be attached to the scalp using, for example, adhesive or another known method, such as using straps or implantation. Preferably, a removable, minimally invasive method is used, such as a semi-permanent adhesive as further described herein to allow for attachment of wearable scalp device 100 to the scalp 4 and maintenance of the wearable device position throughout daily activities for an extended period of time, such as hours, days, weeks, etc. Such adhesive may be easily removable, i.e., solvable in water and/or soap, to allow a user to remove wearable scalp device 100 at will. Preferably, applied adhesive is configured to facilitate conductive activity, e.g., electrochemical communication, to allow for such communication between the scalp and wearable scalp device 100 so that brain activity may be measured and communicated by wearable scalp device 100 as described further herein. Wearable scalp device 100 may be positioned on scalp 4 so that the electronics of the wearable scalp device 100 may obtain data related to at least the occipital lobe, temporal lobe, and/or parietal lobe. In some embodiments, wearable scalp device 100 may not include protrusions, so that housing 102 comes into direct contact with the user's scalp via adhesive or another attachment mechanism as discussed above.

Still referring to FIGS. 2 and 3, the plurality of protrusions 106 are dispersed among the sections 104 of wearable scalp device 100 for generally full coverage and a more secure and full attachment when said protrusions 106 are attached to the user's scalp. As illustrated, the protrusions 106 may be positioned along the bottom edges 108 of the wearable device 100, along the bottom edges, i.e., bottom edge 108a, 108b, 108c of each section 104, and/or spaced apart from the bottom edges 108 of the wearable scalp device 100, i.e., a generally middle portion 112 of wearable scalp device 100. The number of protrusions 106 may vary as desired for sufficient attachment and reading of brain activity. Protrusions 106 may be flexible to allow for increased malleability to facilitate positioning of wearable scalp device 100 on the head, while being rigid enough to support housing 102. Preferably, protrusions 106 have sufficient length to permit contact with the scalp without significant interference from the user's hair.

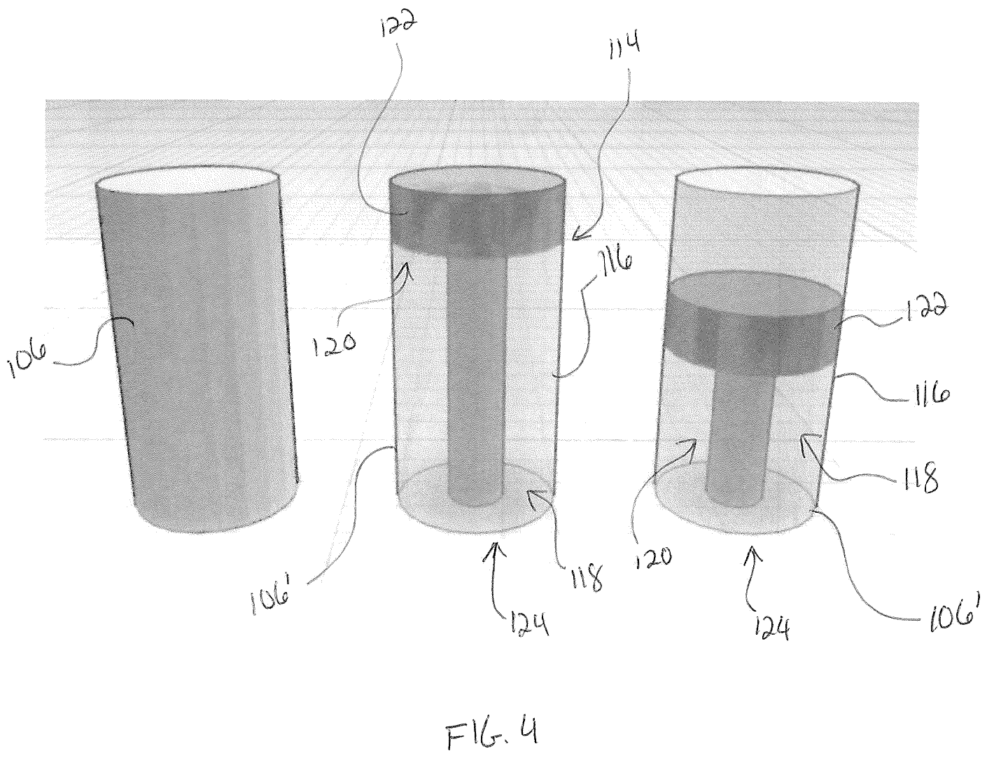

Now referring to FIG. 4, each protrusion 106 may include an applicator 114 for attaching said protrusion 106 to the user. While one or more protrusions 106, or even all protrusions 106, of wearable scalp device 100 may include applicator 114, in some other embodiments, none of protrusions 106 include applicator 114. In protrusions 106 including applicator 114, i.e., protrusions 106′, each of said protrusions 106′ include a sidewall 116 defining a chamber 118 so that protrusion 106′ is hollow. A plunger 120 is positioned within the chamber 118 so that the plunger 120 is axially movable relative to protrusion 106′ while maintaining a fluid-tight fit between a seal 122 of the plunger 120 and an inner surface of sidewall 116. An adhesive may be contained within chamber 118 so that when plunger 120 is activated, the adhesive is extruded through an opening 124 of protrusion 106′ to facilitate attachment of protrusion 106′ to a user's scalp (see, e.g., FIG. 1). The plunger 120 may be activated, for example, by pressing an activation button located either on wearable device 100 or via a separate user interface as described further herein.

In some embodiments, and adhesive solvent may also be housed within chamber 118, so that upon a first activation of plunger 120, the adhesive is applied to the user, and upon a second activation of plunger 120, the adhesive solvent is applied to the user. Application of the adhesive solvent facilitates removal of wearable scalp device 100 from the user while mitigating any potential difficulty or pain associated with removal. Housing of adhesive and associated solvent within protrusion 106′ allows for direct application of said adhesive and/or solvent to the surface onto which wearable scalp device 100 is being applied, improving efficiency of adhesive and solvent use, as well as mitigating any mess associated with application of wearable scalp device 100.

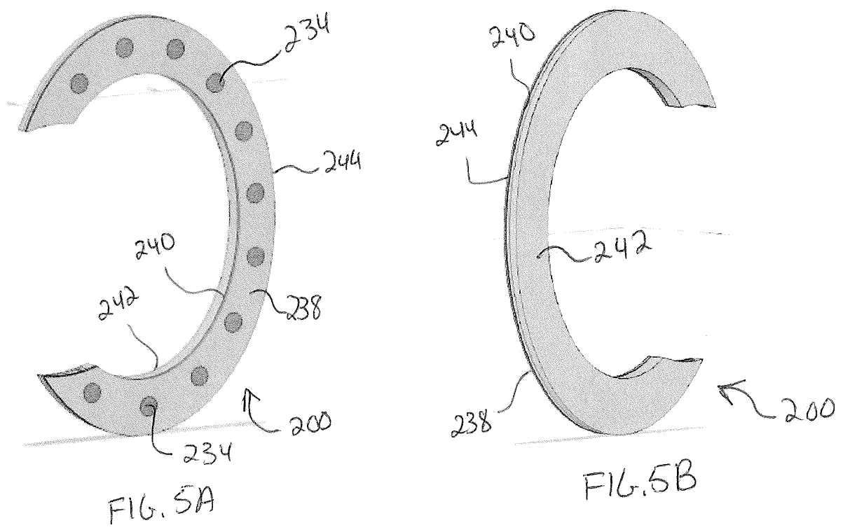

Now referring to FIGS. 5A-5B, a wearable ear device 200 is illustrated, wherein wearable ear device 200 is similar to wearable scalp device 100 except as discussed further herein. Like reference numbers refer to like components.

Wearable ear device 200 is a generally C-shaped structure designed to provide a comfortable fit around the back of the ear, partially surrounding the concha. Wearable ear device 200 may be custom designed and fit for each individual user as discussed further herein. In other occurrences, wearable ear device 200 may be manufactured as a general “one fits all” or “one fits most” mass production product, where the C-shaped structure is configured to comfortable fit around the concha of the general public. Occurrences may exist where a plurality of wearable ear devices are mass produced while another plurality of wearable ear devices are custom designed and produced.

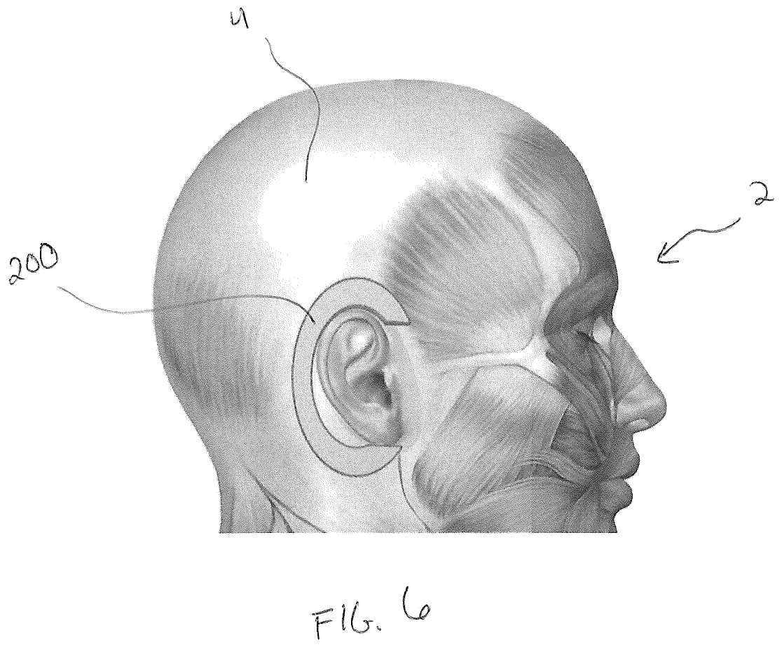

It is understood that the description of wearable ear device 200 as a C-shaped structure is intended to indicate that the shape of the device is curved in a manner to fit in a conforming manner behind a user's ear as shown in FIG. 6. For example, the wearable ear device 200 may be a full C-shape, a half-C-shape, a quarter-C-shape, a three-quarter-C-shape, or another curved shape. It is further understood that the illustration provided in FIG. 6 indicates only that the device conforms to the shape of the ear and that wearable ear device 200 may be more form-fitting to the rear of the ear for a discrete fit.

Wearable ear device 200 has a body 244 comprised of a plurality of layers, including an adhesive layer 238 configured to facilitate attachment of the wearable ear device 200 to a user, an electronics module layer 240, and an outer shell 242. As discussed further herein, each of the electronics module layer 240, the outer shell 242, and the adhesive layer 238 are comprised of a flexible material to facilitate conformation to a user's scalp. A plurality of electrodes 234 are positioned along the body 244 of the wearable ear device so that the electrodes 234 are in communication with the electronics module layer 240 and exposed through the adhesive layer 238 to facilitate contact between the electrodes 234 and the skin of the user.

While the wearable ear device 200 is illustrated as having a plurality of electrodes 234 in FIG. 5A and as described as such above, it is also understood that wearable ear device 200 may have a single electrode 234, or at least one electrode 234. While FIG. 5A illustrates an embodiment having eleven electrodes, a greater or smaller number of electrodes making up a plurality of electrodes as described herein may vary. For example, the plurality of electrodes may include two, three, four, five, six, seven, eight, nine, ten, twelve, thirteen, fourteen, fifteen, sixteen, seventeen, eighteen, nineteen, twenty, or more electrodes in embodiments having a plurality of electrodes. Furthermore, while electrodes 234 are illustrated as being evenly spaced along the entirety of wearable ear device 200, electrodes 234 may be alternately distributed along at least a portion of wearable ear device 200 in other embodiments. For example, electrodes 234 may be distributed along only a portion of wearable ear device 200 in some embodiments. In certain embodiments, electrodes 234 may not be spaced evenly.

Each electrode 234, whether a single electrode or each of a plurality of electrodes, represent at least one custom measurement/communication channel, within each channel being monitored and analyzed as described further herein. Each electrode 234 may be a dry electrode. In other embodiments, each electrode 234 may be a hybrid electrode, such as a microneedle-based electrode or an electrode comprising a foam, hydrogel, or other form of hybrid chemical makeup as known in the art. Each electrode 234 may comprise metallic materials such as silver, gold, platinum, or an alloy thereof; carbon-based materials such as graphene and carbon nanotubes; polymers; or any combination thereof. Other electrode materials as known in the art may also be used. Each electrode 234 is preferably comprised of a biocompatible material.

Adhesive layer 238 comprises an adhesive tape or gel configured to selectively secure the wearable ear device 200 in position when applied to a user. In use, wearable ear device 200 is applied directly to the user's skin, the side with the adhesive layer 238 skin-facing, so that the adhesive layer 238 is placed into direct contact with the user's skin to hold the wearable ear device 200 in position. The adhesive of the adhesive layer 238 may be configured for long-lasting wear, including wear up to 1 hour, up to 6 hours, up to 12 hours, up to 24 hours, up to 2 days, up to 3 days, up to 5 days, up to 1 week, up to 2 weeks, up to 3 weeks, or longer. The adhesive is preferably biocompatible; sweat, oil, and water-resistant; and/or sweat, oil, and water-proof. Once the adhesive is no longer functional, i.e., if the adhesive has worn out, expired, washed out, or is otherwise no longer functional, the tape or gel may be removed and replaced with a new tape or gel adhesive form. In this way, the life of the wearable ear device 200 extends beyond the life of the adhesive and may be reusable past the life of the adhesive. In some embodiments, wearable ear device 200 may include a clip in addition to or as an alternative to adhesive of adhesive layer 238 to facilitate attachment of wearable ear device 200 to the user.

Outer shell 242 is preferably a generally thin layer of material that imparts form to the wearable ear device 200 while maintaining discretion and/or a ductile or flexible characteristic that facilitates a form-fitting attachment of the wearable ear device 200 to the user. The outer shell 242 is preferably formed from a water-resistant or waterproof material to facilitate long term wear by the user while protecting electronics module layer 240 from moisture-related malfunction. The outer shell 242 may further include at least one port (not shown) for receiving a charger to charge an on-board battery described further herein.

Wearable ear device 200 may be custom manufactured using, for example, additive manufacturing. A photo may be taken of the appropriate section of a user's ear and/or scalp and uploaded to an appropriate computer device containing an algorithm or connected to an appropriate web-based service. The algorithm or web-based service may then review the picture and determine the optimal color, size, and/or shape of a preferred wearable ear device 200, taking into account the general size and shape as described above and in view of the size and shape of the electronics module 246 as described further herein, as the electronics module 346 (FIG. 6), and therefore the electronics module layer 240, must be sized and shaped in a manner allowing for housing of the required electronics as described further herein. The algorithm or web-based service may then generate a print file or other file informing the custom manufacturing of the wearable ear device 200 to an appropriate size, shape, and color according to a specified user.

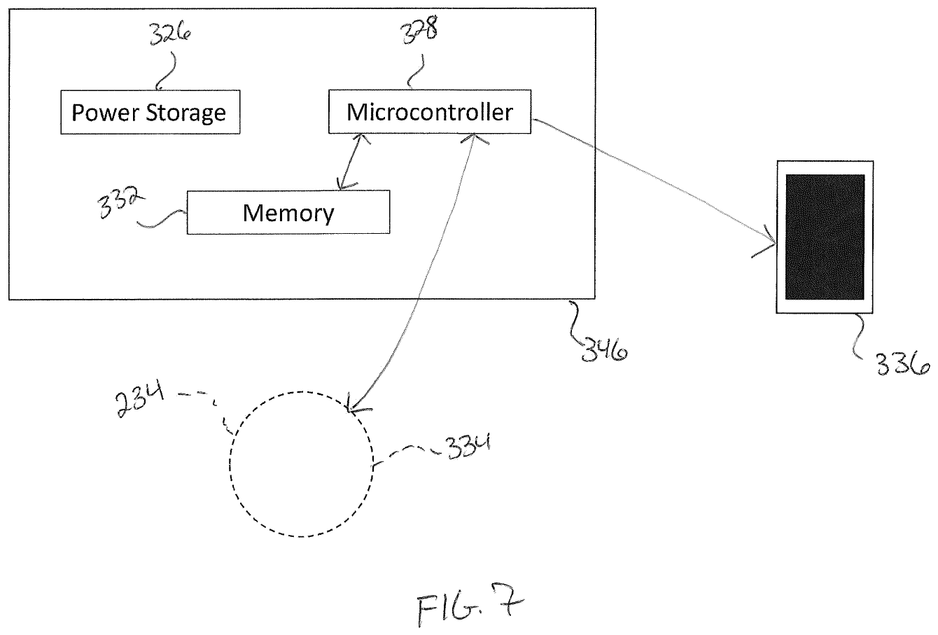

FIG. 7 provides a schematic example of the electronics module 346 wearable scalp device 100 and wearable ear device 200, along with a system 300 including electronics module 346 and associated devices. Electronics module 346 may include, for example, a power storage 326, a microcontroller 328, and a memory 332. The electronics module may further include any combination of at least one of a capacitor, transistor, amplifier, resistor, accelerator, and/or other known electronics components for the proper transmission, reception, and/or processing of a signal as disclosed further herein. In embodiments including an accelerometer, the movement patterns of a wearer may be tracked and analyzed. This data may be used to remove non-brain-related signal from collected data during the pre-processing, feature extraction, and/or analysis processes as disclosed further herein, as well as for characterizing the movements and patterns of the user.

Power storage 326 is preferably a high capacity power storage component, such as a lithium ion battery or other suitable power storage component facilitating long-term use of the associated wearable device 100, 200. For example, power storage 326 may facilitate constant operation of the corresponding wearable device 100, 200 over a period of hours, days, or weeks without necessary charging. In some embodiments, power storage 326 is capable of reach a maximum state of charge with minimal charging time to allow a user to quickly resume usage of the corresponding wearable device 100, 200 after charging is complete. For example, power storage 326 may be capable of reaching a maximum state of charge after approximately an hour or less of charge time.

The microcontroller 328 of wearable device 100 may be in communication with a user interface 336. For example, microcontroller 328 may have Bluetooth® capabilities and/or include a near field communication (“NFC”) module. In other embodiments, microcontroller 328 may include other transmission capabilities—wired or wireless—as known and understood in the art. For example, the microcontroller 328 may be capable of Wi-Fi® connectivity to allow for uploading of data to a cloud server or other remote device. User interface 336 may be a personal electronic device, such as a smart phone, tablet, or computer belonging to the user, a guardian of the user, or a medical provider. In other embodiments, the microcontroller may be in communication with a plurality of user interfaces.

Electronics module 346 may be in an analog configuration. In the analog configuration, electrodes 334 collect brain activity and transmit the collected activity to microcontroller 328, which in turn transmits the raw data corresponding to the collected activity to the user interface 336 for processing as described further herein. In other embodiments, electronics module 346 may be in a neuromorphic configuration. In the neuromorphic configuration, electrodes 334 collect brain activity and transmit the collected activity to microcontroller 328, which processes the raw data corresponding to the collected activity and transmits the processed data to the user interface 336 as described further herein. Before processing, whether the data is being processed by microcontroller 328 or user interface 336, the raw data flows through a pre-processing pipeline of at least one step. At least one feature is extracted from the processed data, and at least one continuous machine learning algorithm or neural network makes a determination about the cognitive function of the user's brain according to the extracted feature as described further herein. The brain activity data collected and processed by electronics module 346 and/or related system 300 may provide a user with a usable insight for tracking and treatment of cognitive function and/or related ailments.

Each electrode 334, that is each electrode for either wearable device 100, 200 may be any one of an electroencephalogram (“EEG”) electrode, an electromyography (“EMG”) electrode, or an electrooculogram (“EoG”) electrode. Wearable devices 100 may include any combination of EEG, EMG, and EoG electrodes, i.e., all EEG electrodes, all EMG electrodes, all EoG electrodes, all either EEG or EMG electrodes, all either EEG or EoG electrodes, all either EoG or EMG electrodes, or a combination of all of EEG, EMG, and EoG electrodes. Each electrode 334 may be either active or passive in nature. In embodiments including active electrodes, each active electrode may include an amplifier.

In systems including some combination of EEG and EoG and/or EMG electrodes, data from each electrode type may be used in conjunction with the other present electrode type(s) to remove non-brain-related signals from the data during pre-processing, feature extraction, and/or analysis processes as described further herein. In addition, data form EMG and/or EoG electrodes may be used to detect patterns in muscle and/or eye movement that represent specific physiological and/or neurophysiological states (i.e., seizures).

A contact sensor (not shown) may be in electrical communication with at least one electrode 334 to continuously monitor contact between the at least one electrode 334 and the user's skin. If insufficient contact is detected, the contact sensor transmits a signal to the microcontroller 328, which in turn sends a signal to user interface 336 so that a user may be notified of the insufficient contact, allowing the user to adjust or reapply the device to ensure sufficient contact and therefore proper tracking and monitoring of neurological and/or cognitive activity as described further herein.

### Processing and Display of Cognitive-Related Data

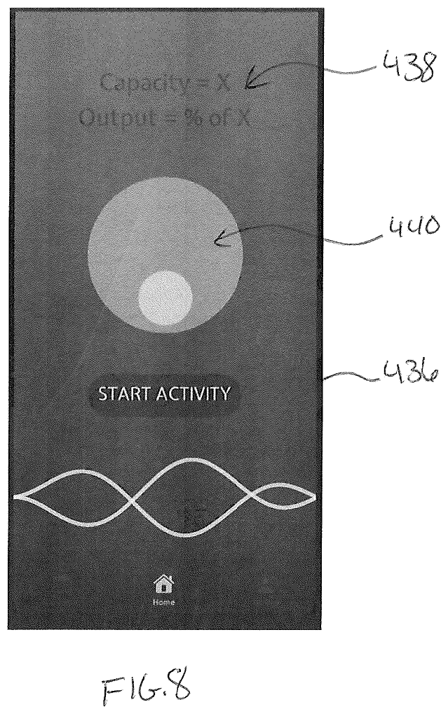

Referring to FIGS. 8-14, a user interface 436 for accessing a mapping of cognitive activity, including cognitive recovery, cognitive output, learning habits, and other information discernable from the brain activity collected by electrodes 334. In multiple embodiments, the user interface may have differing characteristics or visual features. As illustrated, the exemplary user interface may include text communicating the current cognitive capacity and cognitive output of the user.

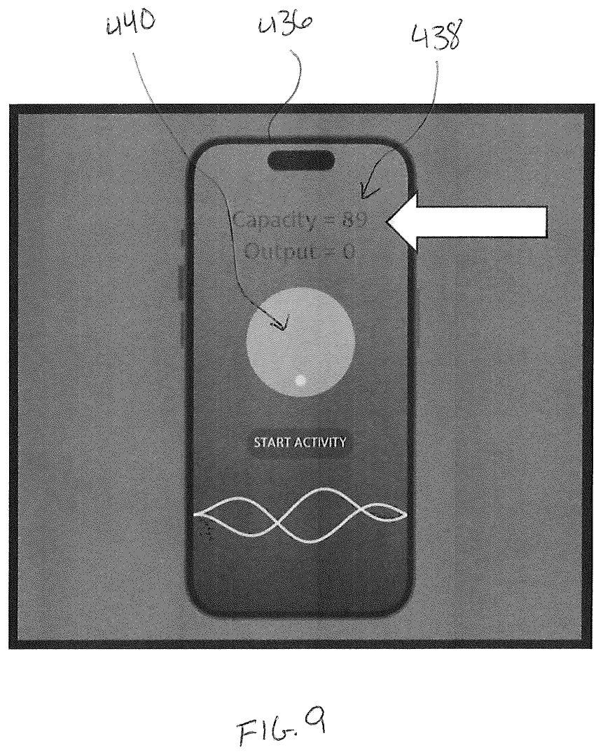

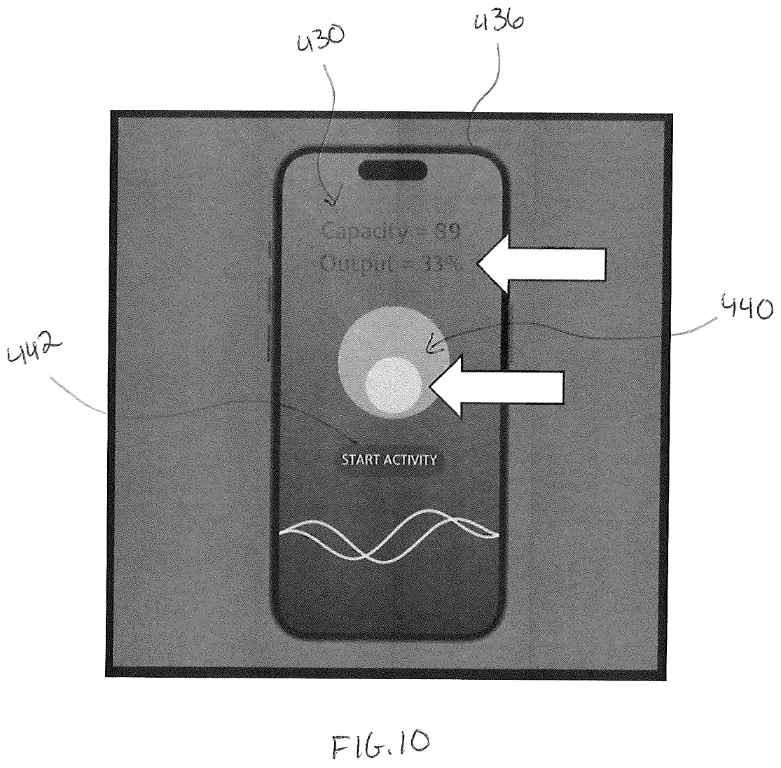

For example, for tracking everyday cognitive function, user interface 436 may provide the user with a snapshot look at the capacity and output of the user's cognitive function during a given event or activity as shown in FIG. 8. The cognitive output may be illustrated in a text format as shown at 438 and/or in a graphical format as shown at 440. For an exemplary day using user interface 436 and associated wearable device 100, 200, FIG. 9 provides an example of the brain capacity and output of someone who is beginning their day. Upon waking, the system 300 has determined that the cognitive recovery during sleep has given the user an available brain capacity of 89%; because the user has not yet begun any activity, the cognitive output is 0%, which is shown both in text format 438 and in graphical format 440. After the user has begun their day, the cognitive output indication raises as shown in FIG. 10, reflected both in text format 438 and in graphical format 440.

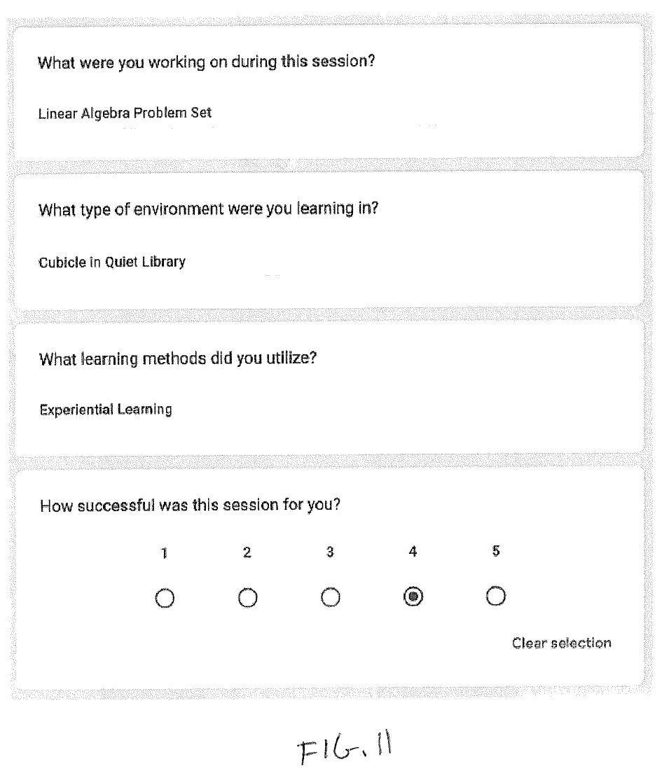

While wearable device 100, 200 and related system 300 may continue this continuous passive cognitive tracking, user interface 436 may also provide a function allowing a user to map cognitive output during a particular activity, as shown at 442 in FIG. 10. For example, if a user is beginning a particularly mentally engaging activity, the user may select the activity indicator 442, which the system 300 will identify and track as a tracked event. Once the activity ends, user interface 436 may provide a questionnaire form for the user to fill out, which allows for tracking of symptoms, environmental effects on cognitive abilities, cognitive output in relation to similar activities, and other trends. A sample questionnaire is illustrated at FIG. 11, although various questionnaires with any number of questions related to cognitive activity may be considered.

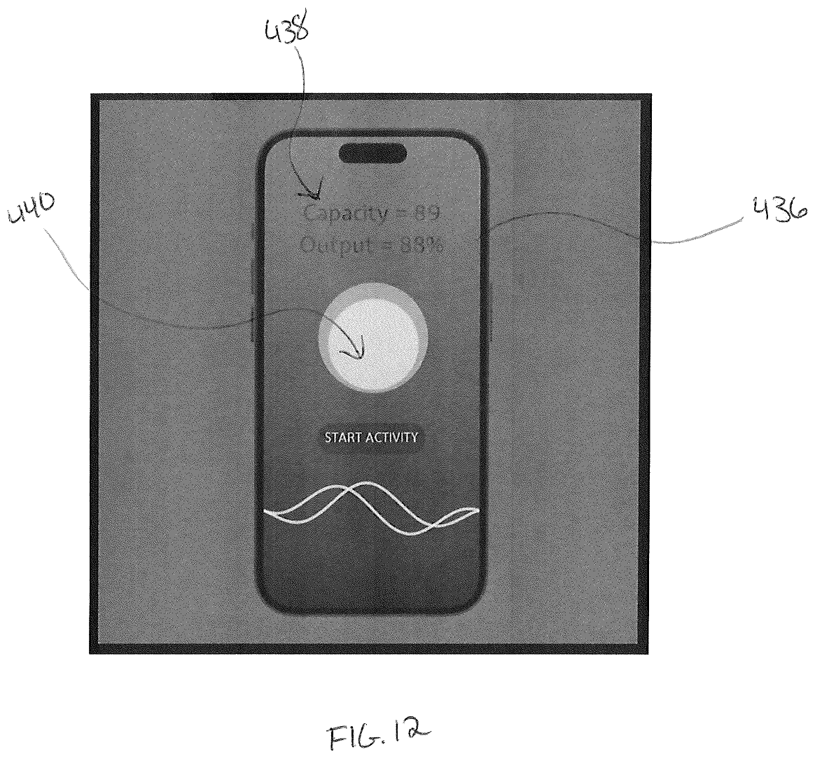

As mentioned above, as the user continues their day, the wearable device 100, 200 and related system 300 continues to track cognitive output in relation to the day's starting brain capacity. For example, FIG. 12 illustrates user interface 436 indicating a cognitive output of 88% in comparison with the starting cognitive capacity of 89%; if the user is at end-of-day, i.e., going to sleep, then this indication may communicate that the user properly allocated his or her cognitive output for the day. However, if the user is not at end-of-day, this indication may communicate to the user that he or she should partake in activities which do not require a large amount of mental processing for the remainder of the day. Referring again to graphical format 444 in FIGS. 8-10 and 12, the larger circle represents the user's capacity, whereas the output is represented by a smaller circle inside the larger. As more brain output is consumed, the smaller circle becomes larger to visualize the cognitive output amount in comparison to the day's beginning cognitive capacity.

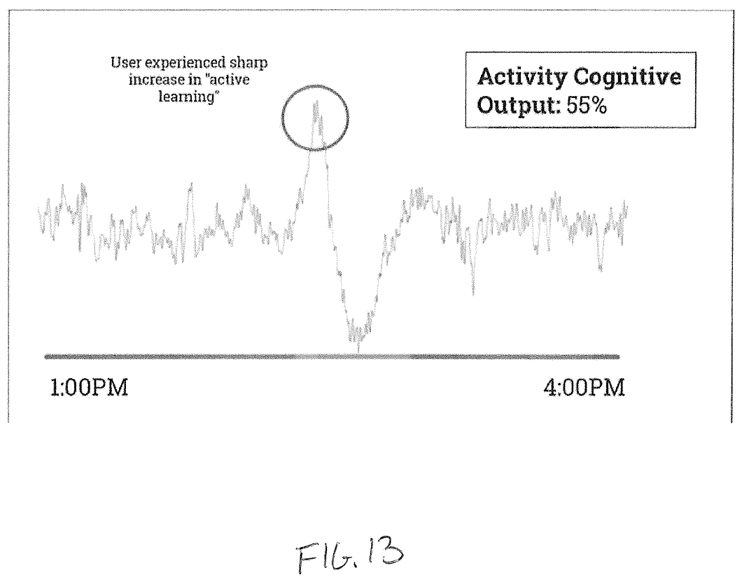

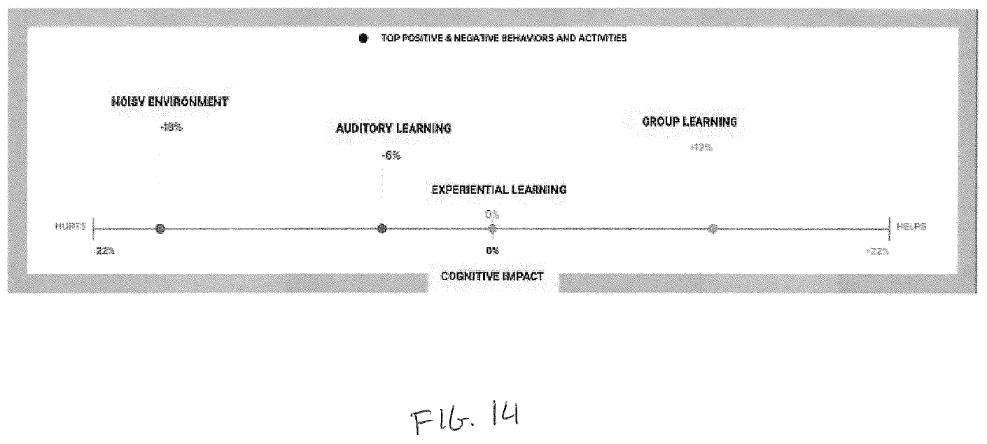

As the user continues to use wearable device 100, 200 in tandem with system 300, the system 300 is able to find and display trends of cognitive capacity and output in relation to certain activities, environments, times of day, etc. For example, as illustrated in FIG. 13, user interface 436 may provide a cognitive output timeline indicating the moments in which the user was required to use the most cognitive energy during the day. As illustrated in FIG. 14, certain trends may also be mapped according to user input and measured cognitive function to provide the user with information on how to efficiently allocate cognitive resources, as well as other beneficial insights.

In other applications, wearable devices 100, 200 and corresponding system 300 may process and export data related to tracking of cognitive-related illnesses, such as epilepsy. In such an application, a related user interface may include tracking options related to seizure alerts, medication tracking, habit tracking, sleep tracking, stress level tracking, and/or any combination of the above.

If system 300 and corresponding wearable device 100, 200 is configured for tracking and alerting a user and/or medical professional of seizure activity, the system 300 monitors neurological and/or cognitive activity, pre-processes the raw data, extracts at least one feature from the processed data, and uses at least one continuous machine-learning algorithm or neural network to make a determination about the neurological and/or cognitive function of the user. Such determinations may include the present occurrence of a seizure event, the likelihood of an impending seizure event, and/or a sleeping event, including the sleep cycle the user is currently experiencing. If the system 300 makes a judgment that there is a present occurrence of a seizure event or a high likelihood of an impending seizure event, the system 300 is configured to transmit a signal from at least one of the microcontroller 328 and the user interface 336 to the user and/or the user's guardian or caretaker and/or medical personnel.

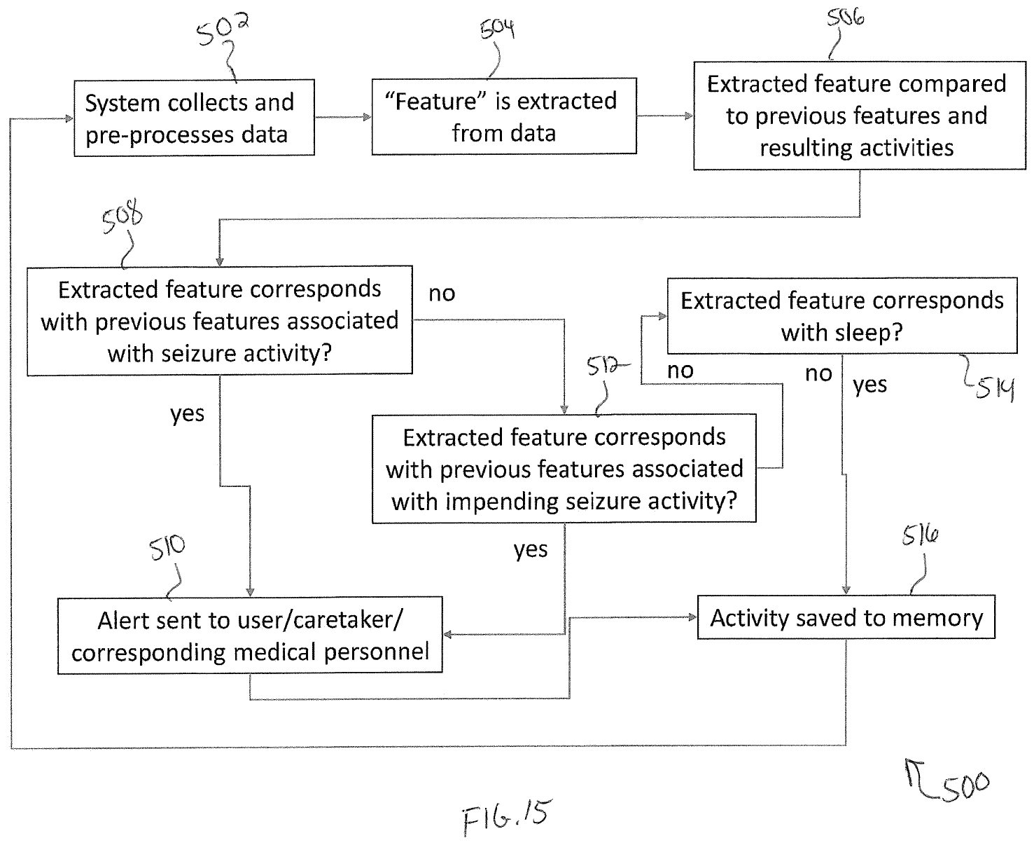

For example, referring to FIG. 15, a method 500 for tracking and reporting seizure activity is illustrated. At box 502, the system 300 collects data related to the neurological and/or cognitive function of the user and pre-processes the collected data. At box 504, a “feature” is extracted from the collected data. While this step refers to “a” feature, it is recognized that this refers to “at least one” feature. Typically, a plurality of features are extracted and analyzed on a continuous basis.

The microcontroller or user interface, depending on whether the system is analog or neuromorphic, respectively, compares the extracted feature to previously identified features and the activities corresponding with the previously identified features to predict an event at box 506. If, at box 508, the system determines the extracted feature corresponds with previously identified features associated with seizure activity, a presently occurring seizure event alert is sent to a user interface of the user, caretaker, and/or corresponding medical personnel at box 510 and the activity is recorded at box 516. If, at box 512, the system determines the extracted feature corresponds with previously identified features associated with impending seizure activity, a seizure warning alert is sent to a user interface of the user, caretaker, and/or corresponding medical personnel at box 510 and the activity is recorded at box 516. If, at box 514, the system determines the extracted feature corresponds with previously identified features associated with sleep and/or a specific sleep cycle, the activity is recorded at box 516. Illustratively, method 500 is a closed, continuous loop which allows for continuous monitoring via wearable devices 100, 200 and associated system 300 as long as the wearable devices 100, 200 are in operation.

Through the machine-based learning nature of the system 300, the microcontroller and/or user interface may determine which electrodes 334 of the system 300 are performing at the highest level of signal quality relative to other electrodes 334 of the system 300. The system 300 may then use this data to weigh the data appropriately during pre-processing, extracting, and analysis stages of method 500 or other applications as described herein. Such weighed consideration may be given in tandem with contact sensors as described above or in absence of such sensors. In other embodiments having contact sensors as described above, data may be weighed accordingly during pre-processing, extracting, and analysis stages of method 500 or other applications as described herein in view of the sufficiency of contact and/or lack of data resulting from lack of contact.

The user interface may also provide questionnaire forms or other user input format to obtain information related to medication dosage, timing of medication consumption, and noted side effects or symptoms. This information is recorded to a memory within the corresponding electronics module and/or another memory associated with the user interface and may be used in tandem with the obtained neurological and/or cognitive activity data obtained from the corresponding wearable device 100, 200 to map any corresponding neurological and/or cognitive data with the input information to determine any statistically significant correlations, causations, or other insights into the effects of the medication on the wearer's neurological and/or cognitive function. Similarly, questionnaire forms or other user input format may be used to track other user habits or activities and the corresponding effects on neurological and/or cognitive function.

As mentioned above, wearable device 100, 200 and corresponding system 300 may also be used to track the user's sleep and sleep cycles according to the monitored neurological and/or cognitive function of the user. The data collected relating to the user's sleep and sleep cycles may also be mapped according to the user's neurological and/or cognitive functions to medically analyze changes in sleep patterns and/or the effects of the user's sleep patterns on the neurological and/or cognitive function of the user.

While the description of cognitive tracking and mapping is provided in view of corresponding figures, the figures provided herein do not limit the described user interface to the ornamental appearance illustrated. In other words, an application configured to determine and/or provide the information as described herein is contemplated by the disclosure.

The terms “first”, “second”, “third” and the like, whether used in the description or in the claims, are provided for distinguishing between similar elements and not necessarily for describing a sequential or chronological order. It is to be understood that the terms so used are interchangeable under appropriate circumstances (unless clearly disclosed otherwise) and that the embodiments of the disclosure described herein are capable of operation in other sequences and/or arrangements than are described or illustrated herein.

The terms “installed”, “provided with”, “sleeved/connected”, “connected”, etc., whether used in the description or in the claims, should be understood broadly. For example, “connected” can be a fixed connection, a detachable connection, or an integral connection, a mechanical connection, an electrical connection, a direct connection, or an indirect connection through an intermediate medium, and it can be a connection between two members. For those of ordinary skill in the art, the specific meaning of the above terms in the present disclosure can be understood under specific conditions. The terms “couples”, “coupled”, “coupler”, and variations thereof are used to include both arrangements wherein two or more components are in direct physical contact and arrangements wherein the two or more components are not in direct contact with each other (e.g., the components are “coupled” via at least a third component, but yet sill cooperate or interact with each other).

While this invention has been described as having exemplary designs, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. A wearable device for measuring neurological function, the device comprising:
a housing;
at least one electrode coupled to the body;
an electronics module coupled to the body and in electric communication with the electrode, the electronics module including a microcontroller;
an adhesive configured to couple the wearable device to a user.

2. The wearable device of claim 1, wherein the wearable device defines a partial C-shape.

3. The wearable device of claim 1, wherein the housing is comprised of at least three circular sections and each one of the at least three circular sections houses an electrode.

4. The wearable device of claim 1, further comprising a plurality of prongs distributed along a bottom surface of the wearable device.

5. The wearable device of claim 4, wherein at least one of the plurality of prongs defines a chamber containing a plunger and a quantity of adhesive, the plunger configured to extrude the quantity of adhesive through an opening defined by the prong to facilitate attachment of the wearable device to the user.

6. The wearable device of claim 1, wherein the wearable device includes at least three electrodes.

7. The wearable device of claim 1, wherein the housing defines an outer shell layer, the adhesive defines an adhesive layer comprised of a gel or adhesive tape, and the electronics module defines an electronics module layer sandwiched between the adhesive layer and the outer shell layer.

8. The wearable device of claim 1, wherein the adhesive is configured to maintain contact between the wearable device and the user for at least one week.

9. A system for measuring neurological function, the system comprising:
a wearable device including an electronics module having a microcontroller; and
a user interface;
wherein the microcontroller is configured to:
receive data corresponding to neurological or cognitive activity of a user, the data collected by at least one electrode;
compare the received data with previously saved data to determine a status of the user;
transmit the received data to the user interface.

10. A method for identifying a seizure occurrence, the method comprising:
collecting neurological data of a user with an electrode of a wearable device;
extracting a neurological feature from the data with a microcontroller;
comparing the extracted feature to previously identified features with at least one of the microcontroller or a user interface;
determining, with the at least one of the microcontroller or the user interface, if the extracted feature corresponds with a seizure occurrence.
